(19) United States
(12) Patent Application Publication
(10) Pub. No.: US 2023/0070370 A1
(43) Pub. Date: Mar. 9, 2023

(54) PROCESS FOR THE PREPARATION OF PANOBINOSTAT

(71) Applicants: FLAMMA SPA, Chignolo d'Isola (BG) (IT); UNIVERSITÀ DEGLI STUDI DI PAVIA, Pavia (PV) (IT)

(72) Inventors: Massimiliano ANDREOLI, Goito (MN) (IT); Teresa BASILE, Acireale (CT) (IT); Alessio PORTA, Certosa di Pavia (PV) (IT); Giuseppe ZANONI, San Genesio ed Uniti (PV) (IT); Roberto FANELLI, Bollate (MI) (IT); Massimo VERZINI, Caldiero (VR) (IT)

(73) Assignees: FLAMMA SPA, Chignolo d'Isola (BG) (IT); UNIVERSITÀ DEGLI STUDI DI PAVIA, Pavia (PV) (IT)

(21) Appl. No.: 17/799,106

(22) PCT Filed: Feb. 25, 2021
(86) PCT No.: PCT/EP2021/054683

§ 371 Date: Aug. 11, 2022

(30) Foreign Application Priority Data
Feb. 27, 2020 (IT) 102020000004075

(51) Int. Cl. C07D 209/14; B01J 27/14

(52) U.S. Cl. CPC C07D 209/14; B01J 27/14

(57) ABSTRACT

The present invention relates to a new process for the preparation of Panobinostat and intermediates thereof.

### DESCRIPTION

### Technical Field

The present invention relates to a new process for the preparation of (2E)-N-hydroxy-3-[4-({[2-(2-methyl-1 H-indol-3-yl)ethyl]amino}methyl)phenyl]prop-2-enamide of formula (I), also known as Panobinostat, an active ingredient (as lactate anhydrous) developed by Novartis Pharmaceuticals under the drug brand name Farydak®, for the treatment of patients with multiple myeloma who have received at least two previous regimens, including bortezomib and an immunomodulatory agent.

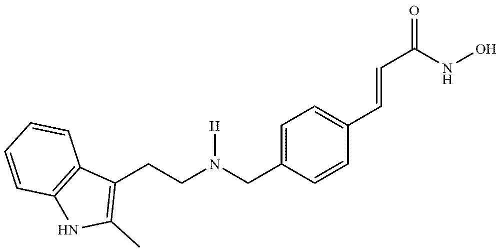 [SMILES: Cc1[nH]c2ccccc2c1CCNCc1ccc(/C=C/C(=O)NO)cc1]

### Background of the Invention

Panobinostat was first described in the International patent application WO02/022577. This patent application discloses also the use of the compound as histone acetylase inhibitor for the treatment of cell proliferative diseases. WO02/022577 describes the synthesis of Panobinostat by means of a reductive amination reaction between 2-methyltryptamine hydrochloride or its free base, and the 4-formylcinnamic methyl ester in the presence of NaBH₃CN, NaBH₄ or H₂ in the presence of Pd/C in MeOH.

2-Methyltryptamine is prepared by reacting 2-methylindole with oxalyl chloride and then with aqueous ammonia to afford the corresponding 2-methylindole-3-glyoxylamide which is subsequent reduced to the amine using LiAlH₄ in dry THF. In another embodiment of WO02/022577, 2-methyltryptamine is obtained by three steps sequence starting with the reduction of 2-chloro-1-(2-methylindol-3-yl)ethanone with BF₃·Et₂O and Et₃SiH in MeCN to afford 3-(2-chloroethyl)-2-methylindole. SN2 chloride displacement with K-phthalimide in DMF at 85° C. and subsequent reaction with MeNH₂ in EtOH/H₂O at 85° C., followed by ethanolic HCl afford 2-methyltryptamine hydrochloride.

In the International patent application WO2007/146718, 2-methyltryptamine is prepared by Fischer indole synthesis starting from phenylhydrazine and 5-chloro-2-methyl-2-pentanone as a carbonyl partner.

Chen et al., Journal of Chemical Research 2018, vol. 42, pages 471-473 describes the synthesis of Panobinostat in a two step procedure starting from 4-(chloromethyl)benzaldehyde and reacting with 2-methyltryptamine via Wittig-Horner reaction in the presence of DBU, DMF and with a large excess of diethylphosphonoacetate. Intermediate (E)-methyl 3-[4-({[2-(2-methyl-1 H-indol-3-yl)ethyl]amino}methyl)phenyl]acrylate is subsequently converted into Panobinostat via nucleophilic substitution reaction with hydroxylamine hydrochloride in the presence of a suitable base.

### DEFINITIONS

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference; thus, the inclusion of such definitions herein should not be construed to represent a substantial difference over what is generally understood in the art.

The terms “approximately” and “about” herein refer to the range of the experimental error, which may occur in a measurement.

The term “room temperature” herein refers to a temperature between 15° C. and 25° C.

The term “halogen” refers herein to fluorine (F), chlorine (Cl), bromine (Br), or iodine (I).

The term “C₁-C₆ alkyl” herein refers to a branched or linear hydrocarbon containing from 1 to 6 carbon atoms. Examples of C₁-C₆ alkyl groups include but are not limited to methyl, ethyl, n-propyl, isopropyl, n-butyl, sec-butyl, isobutyl, tert-butyl, n-pentyl, n-hexyl.

The term “aryl” herein refers to aromatic mono- and poly-carbocyclic ring systems, wherein the individual carbocyclic rings in the poly-carbocyclic ring systems may be fused or attached to each other via a single bond. Suitable aryl groups include, but are not limited to, phenyl (Ph), benzyl (Bn), naphthyl and biphenyl.

The terms “comprising”, “having”, “including” and “containing” are to be construed open-ended terms (i.e. meaning “including, but not limited to”) and are to be considered as providing support also for terms as “consist essentially of”, “consisting essentially of”, “consist of” or “consisting of”.

The terms “consist essentially of”, “consisting essentially of” are to be construed as semi-closed terms, meaning that no other ingredients which materially affects the basic and novel characteristics of the invention are included (optional excipients may thus included).

The terms “consists of”, “consisting of” are to be construed as closed terms.

### SUMMARY OF THE INVENTION

The invention relates to a novel and efficient process that leads to Panobinostat, which is convenient for the industrial scale and provides the desired product in good yields.

The process of the invention is described in Scheme A.

The following abbreviations are used:

Pd(OAc)₂ = Palladium(II) acetate; P(o-tol)₃ = Tris(o-tolyl)phosphine; Boc = Tert-butyloxycarbonyl; DIEA = N,N-Diisopropylethylamine; THF = tetrahydrofuran; TFA = trifluoroacetic acid; DCM = dichloromethane; NaBH(OAc)₃ = sodium triacetoxyhydroborate; r.t. = room temperature.

Scheme A shows the process for the preparation of Panobinostat characterized by a convergent inverse reductive amination between indole-derivative aldehyde and benzylic amine in which the key hydroxamic moiety has been already installed. Preferably, the final product is isolated as lactate salt.

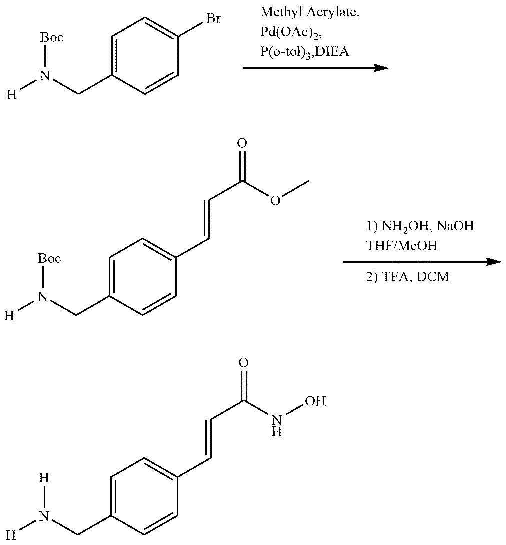

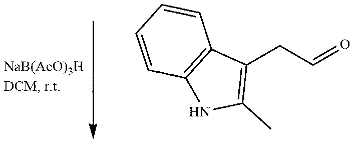

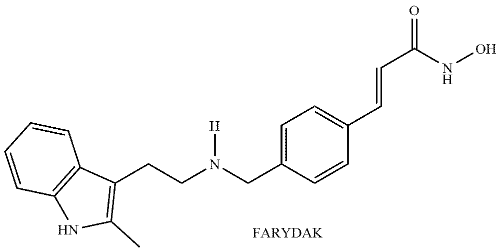

This process is a notable improvement with respect to the prior art and its advantages are summarized below.

Starting from easy-to-prepare or commercially available reagents, tert-butyl 4-((E)-2-(methoxycarbonyl)vinyl)benzylcarbamate is obtained in high yield and with a very low molar loading of the palladium catalyst. The reaction with hydroxylamine hydrate to obtain tert-butyl 4-((E)-2-(hydroxycarbamoyl)vinyl)benzylcarbamate is performed at room temperature, as well as the reductive amination to obtain panobinostat.

### DETAILED DESCRIPTION OF THE INVENTION

According to a first aspect, the present invention relates to a new process for preparing (2E)-N-hydroxy-3-[4-({[2-(2-methyl-1 H-indol-3-yl)ethyl]amino}methyl)phenyl]prop-2-enamide of formula (I), preferably as lactate salt,

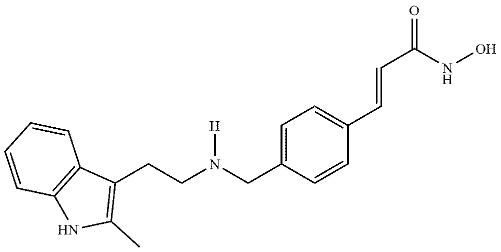 [SMILES: Cc1[nH]c2ccccc2c1CCNCc1ccc(/C=C/C(=O)NO)cc1]

which comprises the steps of:
a) reacting a compound of formula (II)

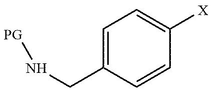

wherein PG is an amino protecting group and X is halogen or an activating group, with an alkyl or an aryl acrylate of formula (III) 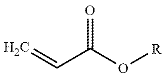
in which R is selected from Me, Et, t-Bu, iPr, Ph or Bn to obtain a compound of formula (IV) 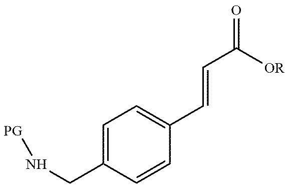
wherein PG and R are as defined above;
b) reacting a compound of formula (IV) with NH₂OH (V) and further deprotection of the amino protecting group to obtain a compound of formula (VI)

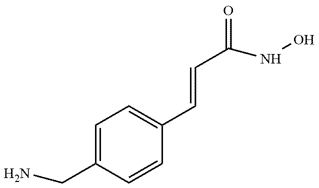 [SMILES: NCc1ccc(/C=C/C(=O)NO)cc1]

c) reacting the compound of formula (VI), preferably in the form of the trifluoroacetic acid salt, with a compound of formula (VII)

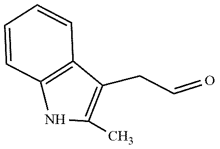

to obtain the compound of formula (I) and
d) optionally, salifying the compound of formula (I) with lactic acid to form the corresponding lactate salt.

In a preferred embodiment of the process, the amino protecting group PG is selected from tert-Butyloxycarbonyl (Boc), Carbobenzyloxy (Cbz), Benzoyl (Bz), Allyloxycarbonyl (Alloc), p-Nitrocinnamyloxycarbonyl (Noc), 2,2,2-Trichloroethoxycarbonyl (Troc), Propargyloxycarbonyl (Poc), Acetyl (Ac), or 2-Trimethylsilylethanesulfonyl (SES).

In a preferred embodiment of the process, X is halogen, preferably bromine. In another embodiment, X is an activating group, preferably, trifluoromethanesulfonate (OTf) or nonaflate (ONf).

In one preferred embodiment, the compound of formula (II) is obtained by protecting the corresponding primary benzylamine or a salt thereof in a solvent and in the presence of an organic or inorganic base.

Preferably, the solvent is selected from polar, non-polar solvents or mixtures thereof. More preferably, water, DCM, tBuOH, ACN, Ethyl acetate, Dioxane, THF, HFIP, toluene or mixtures thereof.

Preferably, the organic or inorganic base is selected from TEA, DlEA, DBU, DBN, DMAP, NaOH, NaOAc, Na₂CO₃, NaHCO₃.

In another embodiment of the process, the step a) is performed in a solvent, in the presence of a transition metal catalyst, a phosphine-based ligand, and an organic or inorganic base.

Alternative ligands to the phosphine-based ligands that can be used in the present process are NHC or Pincer ligands.

Preferably, the solvent used in the step a) is selected from acetonitrile, DMF, DMSO, Toluene, NMP, DMA or mixtures thereof.

Preferably, the transition metal catalyst is Pd(OAc)₂.

Preferably, the phosphine-based ligand is P(o-tolyl)₃.

Preferably, the organic or inorganic base used in the step a) is selected from TEA, DIEA, DBU, DMAP, NaOH, NaOAc, K₂CO₃, Na₂CO₃.

In a preferred embodiment of the process, the transition metal catalyst used in the step a) is in an amount ranging from about 0.01 to about 0.10 equivalents, the phosphine-based ligand is used in an amount ranging from about 0.02 to about 0.2 equivalents, the organic or inorganic base is used in an amount ranging from about 1.0 to about 2.0 equivalents.

In another embodiment, the step b) is performed at room temperature.

In a preferred embodiment of the process, the reaction with NH₂OH of step b) is performed in a polar solvent or in a mixture of polar solvents, preferably in a molar ratio ranging from 1:2 to 2:1.

Preferably, the polar solvent is selected from water, DMSO, DMF, MeOH, THF, pyridine or mixtures thereof.

In another preferred embodiment of the process, the deprotection of the amino protecting group in step b) is performed in a solvent in the presence of a strong acid, or by thermal decomposition.

Preferably, the deprotection of the amino protecting group is performed in methylene chloride in the presence of trifluoroacetic acid, or in dioxane in the presence of hydrochloric acid 4M.

Preferably, the deprotection phase is performed at room temperature.

In another embodiment, the compound of formula (VII) is obtained from 2-(2-methyl-1H-indol-3-yl)ethanol via IBX oxidation.

2-methyl-1H-indol-3-yl)ethanol is obtained from carboxylic acid reduction of commercially available 2-methyl-1H-indol-3-yl) acetic acid.

In a preferred embodiment of the process, the step c) is carried out in a mixture of polar and non-polar solvents, preferably in a molar ratio ranging from 1:10 to 1:1, in the presence of a reducing agent.

Preferably, the mixture of polar and non-polar solvents is selected from methanol/methylene chloride, methanol/toluene, methanol/IPA, Acetonitrile/methylene chloride, Acetonitrile/toluene, or Acetonitrile/IPA. Preferably, the reducing agent is a boron-based reducing agent. More preferably, sodium triacetoxyborohydride, sodiumborohydride or sodium cyanoborohydride. In another embodiment, the pH of step c) is from 4.8 to 6.2.

### EXAMPLES

### Example 1: Preparation of 2-(2-methyl-1H-indol-3-yl)ethanol

The 2-methyl-1H-indol-3-yl) acetic acid 2 g (1.0 equiv 10.5 mmol) was dissolved in anhydrous THF 10 mL and cooled 0° C. A suspension of LiAlH₄1.6 g (4.0 equiv 42.0 mmol) in THF was added dropwise, and the mixture was stirred at 10° C. After 30 min the LiAIH4 was quenched by adding in this order: H₂O, NaOH (10%) and H₂O (1 g of LiAIH4 = 1 mL of H₂O, 1.5 mL of NaOH). The resulting powder was washed with ethyl acetate and evaporated to obtained a clear oil in 92% yield. The desired product was obtained with 96% of purity (1-H NMR).

¹H NMR (300 MHz, CD₃CN) δ 2.36(s, 3 H); 2.84 (t, J = 6.4 Hz, 1 H); 3.65 (q, J = 6.4 Hz, 1 H) 7.04 ( dd, J = 8.4 Hz, 1.9, 1 H), 7.26 ( d, J = 1.4 Hz, 1 H), 7.48 ( d, J = 8.4 Hz,1 H) 8.93 (br s, 1H) ppm.

¹³ C NMR (75.0 MHz, CD₃CN) δ 120.3,118.5,117.9,117.9,117.5,110.1,62.0,27.6,10.5.

IR (neat) 3401,3055,2934,1622,1585,1462,1433,1338,1300,1239,1239,1154,1138,1108, 1043,1010,863,742.

### Example 2: Preparation of 2-(2-methyl-1H-indol-3-yl)acetaldehyde

IBX 7.4 g (1.1 equiv 27.0 mmol) was added to a solution of 2-(2-methyl-1H-indol-3-yl)ethanol 2 g (11.0 mmol) in DMSO (57 mL). After 6 h, the reaction mixture was diluted with water, the resulting white solid was filtered, and the resulting two layers were extracted with Et₂O (3 x 100 mL). The combined organic layers were dried (Na₂SO₄) and volatiles were removed under reduced pressure. The crude mixture was purified by flash chromatography (n-hexane/ AcOEt 6:4) to give the desired product in 55% yield.

¹H NMR (300 MHz, CD₃CN) δ 2.36 (s, 3 H) 3.7(s, 2 H) 7.02-7.13 (m, 2 H) 7.13-7.41(m, 2 H) 9.1(br s, 1 H) 9.6 (s, 1 H) ppm.

¹³C NMR (75.0 MHz, CD3CN) δ 199.4,135.5,133.9,128.6,119.0,117.3,110.4,101.3,38.9, 10.5

IR (neat) 3397,1718,1654,1560,1508,1460,130,3743.

### Example 3: Preparation of Tert-butyl 4-bromobenzylcarbamate

To a solution of 4-bromobenzylamine hydrochloride 1 g (1 equiv 5.0 mmol) in anhydrous DCM (10 mL), Et₃N 1.25 mL (2.0 equiv 9.0 mmol) was added followed by di-tert-butyl dicarbonate 1 mL (1.0 equiv 0.005 mol). The reaction mixture was stirred at room temperature under N₂ overnight.

The reaction was quenched with water (10 mL) and the resulting mixture was extracted with DCM (100 mL x 3). The combined organic layers were washed with brine, dried over Na₂SO₄, filtered, and concentrated under reduced pressure. The desired product was obtained in 91% yield. M.p.80° C.

¹H NMR (300 MHz, CDCl₃) δ 1.47 (s, 9 H) 4.27 (s, 2 H) 4.9 (br s, 1 H) 7.17 (d, J=0.5 Hz 2 H) 7.4 (d, J=0.5 Hz 2H) ppm.

¹³ C NMR (75.0 MHz, CD₃CN) δ 155.7,137.9,131.5,129.0,121.0,77.3,77.1,76.9,43.9,28.2.

IR (neat) 3364,2921,2360,1682,1455,1377,1246,1169,1068,1049,1011,879,839,810, 781,722

### Example 4: Preparation of Tert-butyl 4-((E)-2-(methoxycarbonyl)vinyl) benzylcarbamate

To a solution of tert-butyl 4-bromobenzylcarbamate 0.6 g (1.0 equiv 2.0 mmol) in MeCN ( 3 ml ), methyl acrylate 152 ∟L (1.2 equiv), P(o-tolyl)₃ 0,026.00 g (2% mol), DIEA 0.286 mL (1.2 equiv), and Pd(OAc)₂ 0.013 g (1% mol) were added and the resulting mixture was stirred under N₂ at room temperature for 10 min, the temperature was increased at 100° C. and then stirred for 6.5 h.

After cooling, H₂O (100 mL) was added, and the mixture was extracted with AcOEt (100 mL x 3). The combined organic layers were washed with brine, dried (Na₂SO₄), filtered, and concentrated under reduced pressure.

The crude product was purified through flash chromatography (n-hexane/ AcOEt 8/2) to give the desired product in 67% yield.

M.p.90° C.

¹H NMR (300 MHz, CD₃CN) δ 1.42 (s, 9 H) 3.77 (s, 3 H) 4.25 (d , J=0.3 Hz 2 H) 5.78 (br s, 1 H) 6.5 (d, , J=0.8 Hz 1 H) 7.21 (d, , J=0.5 Hz 2 H) 7.6 (d, , J=0.5 Hz 2 H) 7.6 (d, , J=0.8 Hz 1 H) ppm.

¹³C NMR (75.0 MHz, CD₃CN) δ 169.7,146.8,135.7,130.9,130.2,120.2,120.0,53.8,46.1, 30.2.

IR (neat) 3333,2920,2359,1714,1687,1681,1651,1469,1455,1366,1285,1169,1040,985, 955,935,869,815,723.

### Example 5: Preparation of Tert-butyl 4-((E)-2-(hydroxycarbamoyl)vinyl) benzylcarbamate

Hydroxylamine (20 mL of 50% aqueous solution) was added to a solution of tert-butyl 4-((E)-2-(methoxycarbonyl)vinyl)benzylcarbamate 1 g (1.0 equiv 3.0 mol) in 20 mL of MeOH/THF (1:1). The resulting mixture was stirred at room temperature for 12 h. The reaction mixture was then poured onto ice/6M HCl (50 mL), extracted with DCM (1 x 20 mL) and EtOAc (2 x 20 mL). The combined organic layers were washed with brine (50 mL), dried over Na₂SO₄ and filtered. The resulting solution was concentrated in vacuo to afford a colorless oil, which was triturated with ether until crystallization occurred. The solid was isolated by filtration to give the desired product in 75% yield. M.p.155° C.

¹H NMR (300 MHz, DMSO) δ 1.38 (s, 9 H) 4.11 (d, J=0.03 Hz 2 H) 6.56 (d, J=0.08 Hz 1 H) 7.24 (d, J=0.04 Hz 2 H) 7.43 (m, 2 H) 7.49 (d, J=0.0.04 Hz 2 H) ppm.

¹³C NMR (75.0 MHz, CD₃CN) δ 162.7,155.7,141.6,138.0,133.2,127.4,118.5,77.8, 43.1,28.2.

IR (neat) 2927,1682,1460,1376,1155,1047,967,722.

### Example 6: Preparation of (E)-3-(4-(aminomethyl)phenyl)-N-hydroxyacrylamide ammonium trifluoroacetate salt.

The tert-butyl 4-((E)-2-(hydroxycarbamoyl)vinyl)benzylcarbamate 0.4 g (1.3 mmol ) was added to a solution of TFA 0,524.00 mL (5.0 equiv 7.0 mmol) in DCM (13 mL) and stirred for 1 h at room temperature. Volatiles (TFA and DCM) were removed under reduced pressure to obtain a solid product in quantitative yield.

M.p.160° C.

¹H NMR (300 MHz, DMSO) δ 4.05 (d, J=0.03 Hz 2 H) 4.43 (br s, 1 H) 6.49 (d, J=0.08 Hz 1 H) 7.45 (m, 4 H) 7.5 (d, J=0.08 Hz 1 H) 8.23 (s, 2 H) ppm.

¹³C NMR (75.0 MHz, CD₃CN) δ 162.4,158.4,158.0,137.56,135.1,134.9,129.3,127.6, 119.7,41.9.

IR (neat) 2920,1778,1681,1651,1557,1520,1505,1455,1378,1199,1065,1005,972, 854,827,798,696.

### Example 7: Preparation of Panobinostat

Solution of 2-(2-methyl-1H-indol-3-yl)acetaldehyde 2 g (1.0 equiv 11.0 mmol) and (E)-3-(4-(aminomethyl)phenyl)-N-hydroxyacrylamide ammonium trifluoroacetate salt 2.6 g (1.2 equiv 13.0 mmol) in MeOH/DCM 0.1 M was stirred at room temperature for five minutes. The pH of the reaction mixture is buffered around ⅚ by addition of solid NaHCO₃.

NaB(OAc)₃H (3 equiv, 33.0 mol,) was added and the resulting reaction mixture was stirred for 12 h.

The reaction mixture was concentrated in vacuo, after solubilization of solids with water was filtered through a reverse phase silica pad by washing with methanol. The filtrate was collected and volatiles were removed in vacuo to afford an oil, which until crystallization with EtOAc and MeOH 9:1 give the final product in 45% yield. The desired product was obtained with 88% of purity determined by UHPLC-LC-MS analysis.

M.p.115° C.

¹H NMR (300 MHz, DMSO) δ 2.7 (dd, J=0.03 Hz 0.06, 4 H) 2.30 (s, 3 H) 3.72 (s, 2 H) 6.4 (d, J=0.0.08 Hz 1 H) 6.90 (m, 2 H) 7.19 (d, J=0.04 Hz 1 H) 7.21 (d, J=0.04 Hz 2 H) 7.42 (t, J=0.04 Hz 3 H) 10.65 (s, 1 H) ppm.

¹³C NMR (75.0 MHz, CD₃CN) δ 166.1, 140.7, 137.8,137.5, 134.2,131.8, 129.7,129.5,122.1,120.2,118.2,111.9,105.88,52.1,22.5,11.5.

### Example 8: Preparation of Panobinostat Lactate

DL-Lactic acid 0,042.00 mL (1.0 equiv 573.0 mmol) was added to a solution of Panobinostat 0.2 g (1.0 equiv 573.0 mmol) in 7:3 acetone/water (1 mL). The resulting suspension was heated at 50° C. under vigorous stirring for 1 h to form a clear solution.

The solution was cooled at 10° C., filtered, dried under vacuum to obtain the final product in 89% yield.

The process of the invention lays on a convergent reductive amination between indole-derivative aldehyde and benzylic amine in which the key hydroxamic moiety has been already installed. The latter has been synthesized via a low palladium catalytic loading, 1% mole, Heck reaction which afforded N-BOC protected 4-bromobenzylcarbamate in 67% isolated yield.

The experiments performed on palladium catalyst shown that using the same conditions in each entry changing only the catalyst loading, the desired product in 67% of yield was obtained using only 1.0 % mol of catalyst. The experiments performed are summarized in the table below.

Heck reaction optimization

| Temperature (°C) | Time | Pd Catalyst | Yield% |
|---|---|---|---|
| 100 | 12 h | 5.0 % mol | 65% |
| 100 | 12 h | 3.0 % mol | 61% |
| 100 | 12 h | 2.0 % mol | 63% |
| 100 | 12 h | 1.0 % mol | 67% |

Moreover, hydroxamic acid formation was carried out at room temperature avoiding the use of cryogenic conditions and highly toxic solvents. At this stage of synthetic sequence, no significant impurities have been detected with UHPLC-LC-HESI-MS analysis. In particular, no significant amount of impurity was detected in the TFA salt obtained after BOC deprotection.

The last synthetic step (i.e. the reductive amination) in the Panobinostat synthesis was performed in DCM as a solvent under mild conditions, in particular was used the non-toxic NaB(OAc)₃H as a reductive agent instead of toxic NaBH₃CN used in the prior art. Notably, the reaction was carried out without any carbocation scavenger.

In this step, the most relevant impurities were the residual amine, the selfcondensation product of aldehyde and the Panobinostat oligomerization (in traces amount). However, these impurities were completely removed with standard chromatographic techniques followed by Panobinostat crystallization.

1. A process for the production of (2E)-N-hydroxy-3-[4-({ [2-(2-methyl-1H-indol-3-yl)ethyl]amino}methyl)phenyl]prop-2-enamide of formula (I): 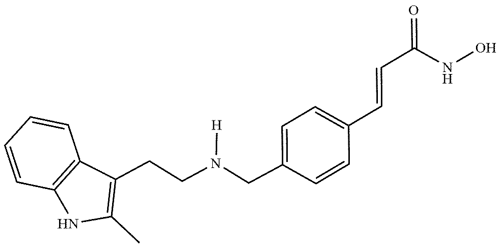 [SMILES: Cc1[nH]c2ccccc2c1CCNCc1ccc(/C=C/C(=O)NO)cc1]
which comprises the following steps:
a) reacting a compound of formula (II) 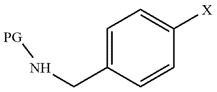
wherein PG is an amino protecting group and X is halogen or an activating group, with an alkyl or an aryl acrylate of formula (III) 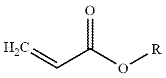
in which R is selected from Me, Et, t-Bu, iPr, Ph, or Bn to obtain a compound of formula (IV) 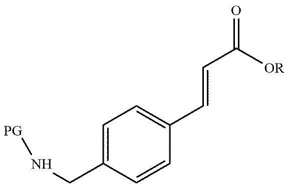
wherein PG and R are as defined above;
b) reacting a compound of formula (IV) with NH₂OH (V) and further deprotection of the amino protecting group to obtain a compound of formula (VI) 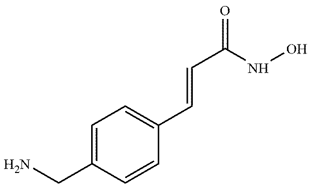 [SMILES: NCc1ccc(/C=C/C(=O)NO)cc1]
c) reacting the compound of formula (VI), preferably in the form of the trifluoroacetic acid salt, with a compound of formula (VII) 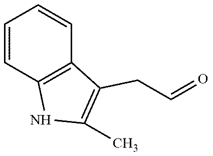
d) optionally, salifying the compound of formula (I) with lactic acid to form the corresponding lactate salt.

2. The process according to claim 1, characterized in that the amino protecting group is selected from Boc, Cbz, Bz, Alloc, Noc, Troc, Poc, Ac, SES.

3. The process according to claim 1, characterized in that the compound of formula (II) is obtained by protecting the corresponding primary benzylamine or a salt thereof in a solvent and in the presence of an organic or inorganic base.

4. The process according to claim 1, characterized in that the step a) is performed in a solvent, in the presence of a transition metal catalyst, a phosphine-based ligand, and an organic or inorganic base.

5. The process according to claim 4, characterized in that the transition metal catalyst is used in an amount ranging from about 0.01 to about 0.10 equivalents, the phosphine-based ligand is used in an amount ranging from about 0.02 to about 0.2 equivalents, the organic or inorganic base is used in an amount ranging from about 1.0 to about 2.0 equivalents.

6. The process according to claim 1, characterized in that the step b) is performed at room temperature.

7. The process according to claim 1, characterized in that the reaction with NH₂OH of step b) is performed in a polar solvent or in a mixture of polar solvents, preferably in a molar ratio ranging from 1:2 to 2:1.

8. The process according to claim 1, characterized in that the deprotection of the amino protecting group in step b) is performed in a solvent in the presence of a strong acid, or by thermal decomposition.

9. The process according to claim 1, characterized in that the compound of formula (VII) is obtained from 2-(2-methyl-1H-indol-3-yl)ethanol via IBX oxidation.

10. The process according to claim 1, characterized in that the step c) is carried out in a mixture of polar and non-polar solvents, preferably in a molar ratio ranging from 1:10 to 1:1, in the presence of a reducing agent.

11. The process according to claim 1, characterized in that the pH of step c) is from 4.8 to 6.2.
